(19) United States
(12) Patent Application Publication
(10) Pub. No.: US 2007/0184098 A1
(43) Pub. Date: Aug. 9, 2007

(54) Esters of flavonoids with w-substituted c6-c22 fatty acids

(76) Inventors: Philippe Moussou, Nancy (FR); Aude Falcimaigne, Nancy (FR); Mohamed Ghoul, Nancy (FR); Louis Danoux, Saulxures Les Nancy (FR); Gilles Pauly, Nancy (FR)

Correspondence Address: COGNIS CORPORATION;PATENT DEPARTMENT, 300 BROOKSIDE AVENUE, AMBLER, PA, 19002, US

(21) Appl. No.: 10/561,551

(22) PCT Filed: Jun. 11, 2004
(86) PCT No.: PCT/EP04/06281

§ 371 Date: Dec. 21, 2006

(30) Foreign Application Priority Data
Jun. 20, 2003 (EP) 03013899.4

(51) Int. Cl. A61K 8/37; A61K 9/127

(52) U.S. Cl. 424/450; 424/451; 424/60; 514/23; 549/400

(57) ABSTRACT

Esters of flavonoids, including flavones, flavonols, flavanones, flavanols, flavanolols, isoflavones, anthocyanins, proanthocyanidins, chalcones, aurones and hydroxycoumarins conjugated by an ester bond to a ω-substituted C6 to C22 fatty acid are provided. The esters may be used in cosmetic, pharmaceutical, and nutritional preparations.

### CROSS-REFERENCE TO RELATED APPLICATIONS

This application is a 35 U.S.C. § 371 filing of International Application No. PCT/EP2004/006281, filed on Jun. 11, 2004, and which claims priority from European application No. EP 03013899.4, filed on Jun. 20, 2003, the entire disclosures of each application are hereby incorporated by reference.

### FIELD OF THE INVENTION

The invention relates generally to esters of flavonoids, and more particularly to ester of flavonoids including flavones, flavonols, flavanones, flavanols, flavanolols, isoflavones, anthocyanins, proanthocyanidins, chalcones, aurones and hydroxycoumarins conjugated by an ester bond to a ω-substituted C6 to C22 fatty acid. In addition it relates to cosmetic, pharmaceutical formulations and nutritional products comprising these flavonoid derivatives and the use thereof.

### BACKGROUND INFORMATION

Flavonoids are a class of natural occurring polyphenols in plants. They are benzo-γ-pyron derivatives and can be classified into several groups (flavones, flavonols, flavanones, flavanols, flavanolols, isoflavones, anthocyanins, proanthocyanidins, chalcones, aurones, hydroxycoumarins) according to the presence of different substituents on the rings and the oxidative degree of ring C (FIG. 1). These flavonoids may also exsist in a glycoside or aglycon form, other modifications such as methylation or acylation of hydroxyl groups increase the diversity of these molecules and their properties.

[image]

For many years, flavonoids have been known for their biological activities. The main properties are their antioxidant activities and enzyme inhibiting activities. They are already used in cosmetic and pharmaceutical formulations for applications associated to various properties such as anti-erythema, anti-blotchiness, sensitive skin, draining, slimming, anti-wrinkles, stimulation of the extracellular matrix, toning up, skin elasticity, anti-ageing, cardiovascular diseases, veinotonic, inflammation, allergy, antiviral, antibacterial properties, stabilizing or protecting therapeutical agents.

For reasons of their anti-radical activity, combined with their absorption spectrum in the UV range, flavonoids may be of interest to prevent photo-oxidative skin damage. UV radiation is one aspect of environmental stress on the skin. The main UV radiation attacking the skin is in the range of 290-320 nm (UVB) reaching the dermis and upper dermis and 320-400 nm (UVA), the most penetrating radiation that affects the dermis. Nuclear or mitochondrial DNA damage, and generation of reactive oxygen species (ROS) which are responsible for lipid and protein damage, are induced by UVA and/or UVB radiation and involve immediate and transient biological responses, for example, inflammation, sunburn, loss of skin elasticity, and delayed and chronic biological responses such as photoaging, or photocarcinogenesis. However, Saija et al. (1998, International Journal of Pharmaceutics, 175, 85), demonstrated that flavonoids were ineffective in formulations.

Moreover, the application of flavonoids in cosmetic, pharmaceutical preparations and nutrition are limited by their low solubility and stability. The solubility of flavonoids (glycosylated and aglycon) in both aqueous phase and lipophilic phase are low. Thus, it is very difficult to incorporate flavonoids in cosmetic, pharmaceutical or nutraceutic formulations. A second drawback is a poor bioavailaibility of flavonoids. Flavonoids are instable due to the presence of many hydroxyl groups in their structure. They are degraded by light, oxygen or oxidizing agents and high temperature.

To improve the UV-protection properties of flavonoids, combination by acylation or alkylation of flavonoids, particularly tiliroside, with aromatic compounds known for their UV-filter properties—for example dibenzoylmethane derivatives or benzoyl derivatives—have been described in International application WO 02/069926. The linking of flavonoids to UV-filter molecules increases the stability of UV-filter. In European application EP 1205475 aglygon flavonoids were also modified with the same UV-filter. These compounds possess the properties of both molecules: the antioxidant and enzyme inhibitor activities of flavonoids and the UV absorption properties of a filter.

In U.S. Pat. No. 4,255,336 derivatives of cyanidan-3-ol with organic carboxylic acid, carbonic acid, sulphonic acid were described in respect of their activity regarding the prevention of hepatic necrosis and lipoperoxydation. These compounds could protect the tissue by the inhibition of the degradation of collagen by collagenase.

Different solutions have been proposed to solve the problem of instability of flavonoids such as encapsulation or addition of antioxidants. Another described way for increasing the stability and the lipophilicity of flavonoids is their acylation with fatty acids by chemical or enzymatic ways. In French patent FR 2706478 therapeutical and cosmetic formulations containing esters of flavanol and procyanolidic oligomers and fatty acid were described. The acylation of phenolic groups has increased the stability of the formulation in respect of color without decreasing the antioxidant activity. In FR 2778663 fatty esters of flavonoids were synthesized chemically. The resulting flavonoid esters were stabilized in preparations and emulsions and their anti-radical activities were preserved. The activity of enzyme inhibition was also increased by the acylation of flavonoids with fatty acids. This is a result of a higher degree of penetration through the cell membrane.

In U.S. Pat. No. 5,844,061 flavonol and procyanolide oligomers were rendered liposoluble and stable by protecting the hydroxyl groups by esterification with fatty acid or aryl acid. The antiradical and antioxidant properties of these esters can be exploited in therapy, cosmetic and dietetic fields.

International patent application WO 00/44757 discloses hydrophilic and lipophilic hesperetin acylated with an organic or inorganic salt of acid or with fatty acid or substituted fatty acid or aromatic acid in order to increase the bioavailability of hesperetin for pharmaceutical application.

The bioavailability of flavonoids may also be improved by increasing their aqueous solubility. Hydrophilic quercetin, apigenin, genistein were obtained by linking a phosphorylated sugar (inositol phosphate) directly or by a short carbon chain (succinate ester). This method increases the aqueous solubility of quercetin due to a linkage with a polar group without diminishing its cytotoxic and antiproliferative activity (WO 96/21440).

In WO 99/63995 the bioavailability of isoflavones was increased by improving their aqueous solubility. This was accomplished by attaching a polar group.

Isoflavones were esterified on an alcohol functionality of aglycon part using a carboxylic acid group or a phosphoric acid group possessing a polar group directly attaching to acid or indirectly linked to a short carbon chain. Succinate, glutarate, adipate and phosphate ester were described as good solubilizers with biological compatibility. Esterified isoflavones can be converted into free isoflavone in biological media by hydrolyzing the ester bond by various enzymes. The esterified isoflavones can be used in nutritional supplements and pharmaceutical preparations as phytoestrogen, antiangiogenic, antioxidant, anticancer, and against ultraviolet skin damage.

Microcapsules of flavonoids have also been obtained by interfacial cross-linking of flavonoids with diacide (FR 2715582). Microcapsules were prepared by mixing an aqueous solution of flavonoid with an organic solution of diacide under vigorous stirring and at elevated pH. The stabilized polyphenol retains its activities.

In German patent application DE 10019235 glycosylated flavonoids and isoflavones acylated with fatty acid or arylaliphatic acid are claimed for cosmetic and pharmaceutical application.

Dicarboxylic acids, having carboxylic groups at the opposite ends of the hydrocarbon chain, represent an interesting class of fatty acid derivatives with bactericidal properties and enzyme inhibition activity. Moreover the majority of these acids are unable to rapidly across liposome membranes. Azelaic acid is already used as cosmetic and therapeutic agent for bleaching of hair, for inhibiting the activity of protease inducing scales and tyrosinase, as anti-acne, antiaging, and as skin lightening agents and have some effects in certain skin disorders.

Accordingly it is an object of the present invention to provide new molecules that combine the properties of flavonoids and ω-substituted C6 to C22 fatty acids with improved biological properties, chemical and physico-chemical stability. These molecules should protect skin, mucus membranes and scalp from damage by UV-radiation and thereby prevent ageing of the skin.

It is another object of the invention to provide formulations comprising these flavonoid derivatives with improved physico-chemical properties and high bioavailability.

### SUMMARY OF THE INVENTION

Briefly described, according to an aspect of the invention, a flavonoid ester with a ω-substituted C6 to C22 fatty acid, where the ω-substituted C6 to C22 fatty acid is a saturated or unsaturated, linear or branched aliphatic C6 to C22—carboxylic acid having one or more polar groups is provided. The flavonoid may be an aglycone or the glycosylated form of a polyphenol selected from a flavone, a flavonol, a flavanone, a flavanol, a flavanolol, an isoflavone, an anthocyanin, a proanthocyanidin, a chalcone, an aurone and a hydroxycoumarin. The polar group may be on the terminal carbon atom of the C6 to C22—carboxylic acid.

In addition, the polar group of the ω-substituted C6 to C22 fatty acid may be a derivative of a carboxylic acid selected from a carboxylic acid (COOH); an amide (CONR′₂ or CONR′₃⁺S^{-) wherein R′ is a hydrogen atom, a saturated or unsaturated, linear or branched alkyl C}1-C6 radical, or an aryl, aralkyl or aralkylene radical and S⁻ is a counter ion; a COHa1 where in Ha1 is a halogen atom; and a COSH. The ω-substituted C6 to C22 fatty acid may also be dicarboxylic, and selected from octanedioic acid, azelaic acid, decandioic acid, dodecandioic acid, hexadecandioic acid and octadecandioic acid. The dicarboxylic acid may also be linked to a flavonoid by an ester bond on one of its carboxylic groups (HOOC—X—C(═O)—O-flavonoid), where X is a saturated or unsaturated, linear or branched alkyl radical (C₄-C₂₀). The ω-substituted C6 to C22 fatty acid may be 11-mercaptoundecanoic acid or thioctic acid, and the polar group of the ω-substituted C6 to C22 fatty acid may be a thiol or an alkylthioalkyl group. The ω-substituted C6 to C22 fatty acid may have two adjacent polar groups selected from diol, dithiol, 1,2-dithiane, 1,3-dithiane and epoxide.

In another aspect of the invention, a nutritional, cosmetic or pharmaceutical composition contains a flavonoid ester described above.

In another aspect of the invention, a nutritional, cosmetic or pharmaceutical composition including liposomes or microcapsules contains a flavonoid ester described above. The nutritional or cosmetic or pharmaceutical composition may contain 0.0001 to 10 wt % of the flavonoid ester.

In another aspect of the invention, the flavonoid ester may be incorporated into a cosmetic preparation as an agent to protect skin and scalp against damage caused by UV radiation, mitochondrial or nuclear DNA damage caused by UV radiation, and aging, or as an anti-inflammatory and/or soothing and relieving agent.

In another aspect of the invention, the flavonoid ester may be incorporated into a preparation for stimulating the metabolism and the immune defense of human skin, including defense against oxidative or environmental stress or pollutants, for a dermatological anti-inflammatory care preparation, or for a draining, veinotonic or slimming preparation.

The flavonoid ester may be used in the above-desribed preparations in quantities of 0.0001 to 10 wt % based on the final composition. The flavonoid ester may also be present in the preparations in the form of liposomes or microcapsules.

### DETAILED DESCRIPTION OF THE INVENTION

The present invention relates to flavonoid esters with ω-substituted C6 to C22 fatty acids. In addition it relates to nutritional, cosmetic or pharmaceutical compositions containing these flavonoid esters and compositions wherein these flavonoid esters are incorporated in liposomes or microcapsules.

The invention also relates to the use of flavonoid esters with ω-substituted C6 to C22 fatty acids to protect skin and scalp against damage caused by UV-radiation such as mitochondrial or nuclear DNA damage from skin aging, to protect against oxidative stress, environmental stress or pollutants, or as an anti-inflammatory agent.

Surprisingly it has been found that the esters of flavonoids with ω-substituted C6 to C22 fatty acids have the property to protect the skin cells against damages caused by UV radiation. As shown in the examples, we have found that the esters of flavonoids according to the invention protect skin cells against UVA and UVB radiation in a more effective manner than the flavonoids alone. Moreover, these esters demonstrated their property to stimulate the GSH metabolism of human skin cells after UVA irradiation, i.e., to stimulate their cellular defenses. They have also anti-inflammatory and soothing properties, as demonstrated by the inhibition of released PGE2 after UVB irradiation.

Thereby these flavonoid esters may be used to protect the skin and scalp and/or to fight against UV and sun damage, erythema, sunburn, mitochondrial or nuclear DNA damage, to prevent or fight photo-aging, providing improvement for signs of ageing as skin wrinkles, elasticity is lost and a decrease in skin thickness.

They may be used also to protect skin, scalp and/or hair shaft and fight against oxidative or stress damages, to protect skin, scalp and/or hair shaft from environmental stress such as pollutants and chemicals.

They may be used to improve the appearance of the skin with local inflammations or microinflammations. Moreover, they may be used to treat sensitive or irritated skin or scalp, as a soothing and anti-itching agent.

Since the flavonoid esters still exhibit the activities of the pure flavonoids the invention allows also their use as anti-free radicals, anti-oxidant, anti-blotchiness agents, for draining treatment, for slimming treatment, for anti-wrinkle treatment, as stimulator of the synthesis of elastin and other extracellular matrix elements, in toning up compositions. They may be used also in compositions for applications related to cardiovascular diseases, veinotonic effect, inflammation disorders, allergy, antiviral and antibacterial properties, stabilizing or protecting therapeutical agents.

The disclosed flavonoid esters show a very good chemical stability. Flavonoid esters with ω-substituted C6 to C22 fatty acids also have a better solubility in lipophilic vehicles, and so they can be easily incorporated in cosmetic, dermatological, pharmaceutical formulations and as nutrional supplements.

Compared to compositions disclosed in International patent application WO 99/63995 the bioavailability of isoflavones was further increased by improving their lipophilic solubility. This was accomplished by attaching not only a polar group, but inserting a C6 to C 22 chain of the fatty acid. Flavonoid esters with ω-substituted C6 to C22 fatty acids can directly be dissolved in the oil phase of the formulations, or totally or partially incorporated in liposomes or microcapsules.

The incorporation in liposomes or microcapsules has the advantage that the release of the active flavonoid esters can be controlled. Especially the disclosed lipophilic flavonoid derivatives are easily incorporated in delivery systems for controlled release. These delivery systems have a very good physico-chemical stability due to the solubility profile of the special flavonoid esters, which also results in an approved bioavailability.

The effective quantity of the disclosed flavonoid esters in formulations is 0.0001 to 10 wt %, preferably 0.001 to 5 wt %, most preferably 0.01 to 2 wt % based on the final composition.

Flavonoids

The term flavonoid represents an aglycone or glycosylated form of the following class of polyphenols chosen from the group consisting of flavones, flavonols, flavanones, flavanols, flavanolols, isoflavones, anthocyanins, proanthocyanidins, chalcones, aurones, hydroxycoumarins. Preferably the glycosylated form is chosen.

Preferably the flavonoids are selected from the group consisting of aglycones or the glycosylated form of kampferol, phloretin, apigenin, luteolin, apigenin, quercetin, hesperetin, naringenin, cyanidin, gossypetin, genistein, daidzein, catechin, epicatechin, fisetin, liquiritigenin and esculetin. More preferably, the flavonoids are selected from the group consisting of the glycosylated forms of quercetin as rutin, glycosylated form of hesperetin as hesperidin, glycosylated form of naringenin as naringin, and glycosylated form of esculetin as esculin.

ω-substituted C6 to C22 Fatty Acids

The term ω-substituted C6 to C22 fatty acid represents a saturated or unsaturated, linear or branched aliphatic carboxylic acid with 6 to 22 carbon atoms having one or more polar group(s)—besides the carboxylic acid group—on carbon atoms anywhere in the chain, preferably at the terminal carbon atom. Preferably these fatty acids have 8 to 18 carbon atoms.

The polar group may be:
(a) a derivative of carboxylic acid chosen from the group consisting of a carboxylic acid COOH; an amide CONR′₂ or CONR′₃⁺S⁻ wherein R′ is a hydrogen atom, a saturated or unsaturated, linear or branched alkyl C1-C6 radical, or an aryl, aralkyl or aralkylene radical and S⁻ a counterion; a COHa1 wherein Ha1 is a halogen atom and a COSH.
   Examples of these ω-substituted C6 to C22 fatty acid group are octanedioic acid, azelaic acid, decandioic acid, dodecandioic acid, hexadecandioic acid, octadecandioic acid.
(b) a thiol or an alkylthioalkyl group such as 11-mercaptoundecanoic acid,
(c) a primary, secondary, tertiary amine or a quaternium salt of hydrogen atom, a saturated or unsaturated, linear or branched alkyl C1-C6 radical, or an aryl, aralkyl or aralkylene radical such as 11-aminoundecanoic acid,
(d) an halogen atom,
(e) a nitro NO₂ group,
(f) an organic or inorganic phosphoric or sulphuric acid,
(g) a hydroxyl group or an alkoxyalkyl group, such as 16-hydroxyhexadecanoic acid, and 12-hydroxystearic acid.

The most preferred derivatives are the derivatives of carboxylic acids (group (a)), especially dicarboxylic acids.

The ω-substituted C6 to C22 fatty acid is also represented by a di-carboxylic acids linked to a flavonoid by an ester bond on one of its carboxylic group, i.e. HOOC—X—C(═O)—O-Flavonoid, wherein X is a saturated or unsaturated, linear or branched alkyl radical (C₄-C₂₀).

The ω-substituted C6 to C22 fatty acid is also represented by a saturated or unsaturated, linear or branched aliphatic chain (C6-C22) having two adjacent polar groups which are diol, dithiol, 1,2 and 1,3 dithiane, and epoxide, such as thioctic acid.

Flavonoid Esters of the Invention

The esters of flavonoids with ω-substituted C6 to C22 fatty acids of the invention correspond to formulas (I) to (X):
Flavone (I): 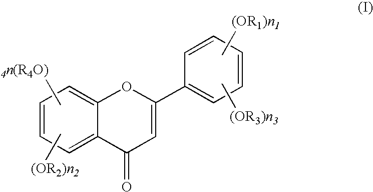
wherein:
(h) the (OR₁), (OR₂), (OR₃) and (OR4) groups are anywhere on the ring,
(i) R₁ and R₂ are identical to or different from each other and represent a hydrogen atom, a saturated or unsaturated, linear or branched alkyl radical (C₁-C₆), a saturated or unsaturated, linear or branched acyl group with 1 to 6 carbon atoms, a monosaccharide or an oligosaccharide,
(j) R₃ and R₄ are identical to or different from each other and comprise a ω-substituted acyl group, or a monosaccharide or an oligosaccharide having at least one or more ω-substituted acyl groups, preferably from 1 to 6 acyl groups and more preferably from 1 to 3 acyl groups,
(k) n₂ and n₃ are identical to or different from each other, are numbers from 0 to 5, and the sum n₁+n₂ does not exceed 5, and
(l) n₂ and n₄ are identical to or different from each other, are numbers from 0 to 4, and the sum n₃+n₄ does not exceed 4.

Examples of flavones are apigenin, luteolol as aglycon form and their glycosylated forms such as diosmin, orientin, saponarin, and shaftoside.

The monosaccharide may be preferably substituted or unsubstituted glucose, rhamnose, galactose, arabinose, and xylose. The oligosaccharide may be preferably the sugar moiety of the following flavonoids: tiliroside, orientin, schaftoside, saponarine, rutin, hesperidin, and diosmin or a polymer of one or more monosaccharide(s) previously described.
Flavonol (II): 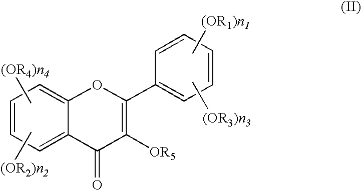
wherein:
(m) the (OR₁), (OR₂), (OR₃) and (OR₄) groups are anywhere on the ring,
(n) R₁ and R₂ are identical to or different from each other and represent a hydrogen atom, a saturated or unsaturated, linear or branched alkyl radical (C₁-C₆), a saturated or unsaturated, linear or branched acyl group with 1 to 6 carbon atoms, a monosaccharide or an oligosaccharide,
(o) R₃, R₄ and R₅ are identical to or different from each other and comprise a co-substituted acyl group, or a monosaccharide or an oligosaccharide having at least one or more ω-substituted acyl groups, preferably from 1 to 6 acyl groups and more preferably from 1 to 3 acyl groups,
(p) n₁ and n₃ are identical to or different from each other, are numbers from 0 to 5, and the sum n₁+n₃ does not exceed 5, and
(q) n₂ and n₄ are identical to or different from each other, are numbers from 0 to 4, and the sum n₂+n₄ does not exceed 4.

Examples of flavonol are kaempferol, quercetin, rhamnetin as aglycon form and their glycosylated form as rutin, quercitrin, hyperoside, and isoquercitrin.

Preferably the monosaccharide may be substituted or unsubstituted glucose, rhamnose, galactose, arabinose, and xylose. Preferably the oligosaccharide may be the sugar moiety of the following flavonoids: tiliroside, orientin, schaftoside, saponarine, rutin, hesperidin, and diosmin or a polymer of one or more monosaccharide(s) previously described.
Flavanone (III): 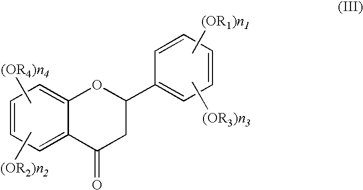
wherein:
(r) the (OR₁), (OR₂), (OR₃) and (OR₄) groups are anywhere on the ring,
(s) R₁ and R₂ are identical to or different from each other and represent a hydrogen atom, a saturated or unsaturated, linear or branched alkyl radical (C₁-C₆), a saturated or unsaturated, linear or branched acyl group with 1 to 6 carbon atoms, a monosaccharide or an oligosaccharide,
(t) R₃, R₄ and R₅ are identical to or different from each other and comprise a ω-substituted acyl group, or a monosaccharide or an oligosaccharide having at least one or more ω-substituted acyl groups, preferably from 1 to 6 acyl groups and more preferably from 1 to 3 acyl groups,
(u) n₁ and n₃ are identical to or different from each other, are numbers from 0 to 5, and the sum n₁+n₃ does not exceed 5, and
(v) n₂ and n₄ are identical to or different from each other, are numbers from 0 to 4, and the sum n₂+n₄ does not exceed 4.

Examples of flavanon are naringenin, eriodictyol, hesperetin, eucalyptin, cirsimaritin, cajaflavanon, hinokiklavon, amentaflavon, bilobetol as aglycon form and their glycosylated form such as hesperidin, neohesperidin, prunin, and naringin.

Preferably the monosaccharide may be substituted or unsubstituted glucose, rhamnose, galactose, arabinose, and xylose. Preferably the oligosaccharide may be the sugar moiety of the following flavonoids: tiliroside, orientin, schaftoside, saponarine, rutin, hesperidin, and diosmin or a polymer of one or more monosaccharide(s) previously described.
Flavonolol (IV): 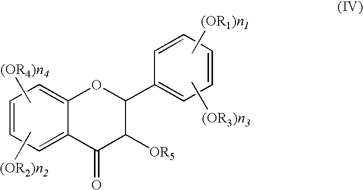
wherein:
(w) the (OR₁), (OR₂), (OR₃) and (OR₄) groups are anywhere on the ring,
(x) R₁ and R₂ are identical to or different from each other and represent a hydrogen atom, a saturated or unsaturated, linear or branched alkyl radical (C₁-C₆), a saturated or unsaturated, linear or branched acyl group with 1 to 6 carbon atoms, a monosaccharide or an oligosaccharide,
(y) R₃, R₄ and R₅ are identical to or different from each other and comprise a ω-substituted acyl group, or a monosaccharide or an oligosaccharide having at least one or more ω-substituted acyl groups, preferably from 1 to 6 acyl groups and more preferably from 1 to 3 acyl groups,
(z) n₁ and n₃ are identical to or different from each other, are numbers from 0 to 5, and the sum n₁+n₃ does not exceed 5, and
(aa) n₂ and n₄ are identical to or different from each other, are numbers from 0 to 4, and the sum n₂+n₄ does not exceed 4.

Examples of flavanolol (also named dihydroflavonol) are fustin, garbanzol, taxifolin, 6-methoxytaxifolin, dihydrokaempferol, dihydrorobinetin as aglycon form and their glycosylated form.

Preferably the monosaccharide may be substituted or unsubstituted glucose, rhamnose, galactose, arabinose, and xylose. Preferably the oligosaccharide may be a sugar moiety of the following flavonoids: tiliroside, orientin, schaftoside, saponarine, rutin, hesperidin, and diosmin or a polymer of one or more monosaccharide(s) previously described.
Isoflavone (V): 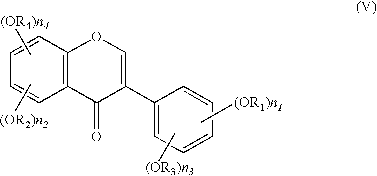
wherein:
(bb) the (OR₁), (OR₂), (OR₃) and (OR₄) groups are anywhere on the ring,
(cc) R₁ and R₂ are identical to or different from each other and represent a hydrogen atom, a saturated or unsaturated, linear or branched alkyl radical (C₁-C₆), a saturated or unsaturated, linear or branched acyl group with 1 to 6 carbon atoms, a monosaccharide or an oligosaccharide,
(dd) R₃ and R₄ are identical to or different from each other and comprise a ω-substituted acyl group, or a monosaccharide or an oligosaccharide having at least one or more ω-substituted acyl groups, preferably from 1 to 6 acyl groups and more preferably from 1 to 3 acyl groups,
(ee) n₁ and n₃ are identical to or different from each other, are numbers from 0 to 5, and the sum n₁+n₃ does not exceed 5, and
(ff) n₂ and n₄ are identical to or different from each other, are numbers from 0 to 4, and the sum n₂+n₄ does not exceed 4.

Examples of isoflavonoids are daidzein, genistein, biochanin A, formonetin, cajanin, prunetin, irigenin, luteone as aglycon form and their glycosylated form as daidzin, genistin, iridin, and puerarin.

Preferably the monosaccharide may be substituted or unsubstituted glucose, rhamnose, galactose, arabinose, and xylose. Preferably the oligosaccharide may be the sugar moiety of the following flavonoids: tiliroside, orientin, schaftoside, saponarine, rutin, hesperidin, and diosmin or a polymer of one or more monosaccharide(s) previously described.
Anthocyanin (VI): 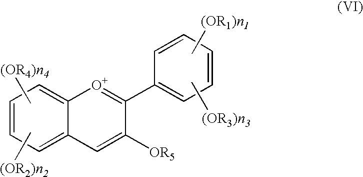
wherein:
(gg) the (OR₁), (OR₂), (OR₃) and (OR₄) groups are anywhere on the ring,
(hh) R₁ and R₂ are identical to or different from each other and represent a hydrogen atom, a saturated or unsaturated, linear or branched alkyl radical (C₁-C₆), a saturated or unsaturated, linear or branched acyl group with 1 to 6 carbon atom, a monosaccharide or an oligosaccharide,
(ii) R₃, R₄ and R₅ are identical to or different from each other and comprise a ω-substituted acyl group, or a monosaccharide or an oligosaccharide having at least one or more ω-substituted acyl groups, preferably from 1 to 6 acyl groups and more preferably from 1 to 3 acyl groups,
(jj) n₁ and n₃ are identical to or different from each other, are numbers from 0 to 5, and the sum n₁+n₃ does not exceed 5, and
(kk) n₂ and n₄ are identical to or different from each other, are numbers from 0 to 4, and the sum n₂+n₄ does not exceed 4.

Examples of anthocyanins are cyanidin, 6-hydroxycyanidin, pelargonidin, okanin, malvidin as aglycon form and their glycosylated form as cyanidin-3-O-galactoside, cyanidin-3-O-rutinoside, pelargonidin, and malvin.

Preferably the monosaccharide may be substituted or unsubstituted glucose, rhamnose, galactose, arabinose, and xylose. Preferably the oligosaccharide may be the sugar moiety of the following flavonoids: tiliroside, orientin, schaftoside, saponarine, rutin, hesperidin, and diosmin or a polymer of one or more monosaccharide(s) previously described.
Chalcone (VII): 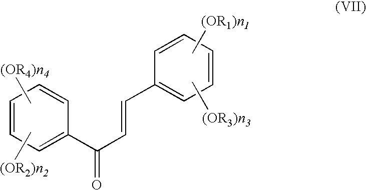
wherein:
(ll) the (OR₁), (OR₂), (OR₃) and (OR₄) groups are anywhere on the ring,
(mm) R₁ and R₂ are identical to or different from each other and represent a hydrogen atom, a saturated or unsaturated, linear or branched alkyl radical (C₁-C₆), a saturated or unsaturated, linear or branched acyl group with 1 to 6 carbon atom, a monosaccharide or an oligosaccharide,
(nn) R₃ and R₄ are identical to or different from each other and comprise a ω-substituted acyl group, or a monosaccharide or an oligosaccharide having at least one or more ω-substituted acyl groups, preferably from 1 to 6 acyl groups and more preferably from 1 to 3 acyl groups,
(oo) n₁ and n₃ are identical to or different from each other, are numbers from 0 to 5, and the sum n₁+n₃ does not exceed 5, and
(pp) n₂ and n₄ are identical to or different from each other, are numbers from 0 to 5, and the sum n₂+n₄ does not exceed 5.

Examples of chalcones are davidigenin, phloretin, isoliquiritigenin as aglycon form and their glycosylated form as phloridzin, and glycyphyllin.

Preferably the monosaccharide may be substituted or unsubstituted glucose, rhamnose, galactose, arabinose, and xylose. Preferably the oligosaccharide may be the sugar moiety of the following flavonoids: tiliroside, orientin, schaftoside, saponarine, rutin, hesperidin, and diosmin or a polymer of one or more monosaccharide(s) previously described.
Aurone (VIII): 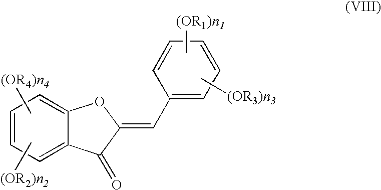
wherein:
(qq) the (OR₁), (OR₂), (OR₃) and (OR₄) groups are anywhere on the ring,
(rr) R₁ and R₂ are identical to or different from each other and represent a hydrogen atom, a saturated or unsaturated, linear or branched alkyl radical (C₁-C₆), a saturated or unsaturated, linear or branched acyl group with 1 to 6 carbon atom, a monosaccharide or an oligosaccharide,
(ss) R₃ and R₄ are identical to or different from each other and comprise a ω-substituted acyl group, or a monosaccharide or an oligosaccharide having at least one or more ω-substituted acyl groups, preferably from 1 to 6 acyl groups and more preferably from 1 to 3 acyl groups,
(tt) n₁ and n₃ are identical to or different from each other, are numbers from 0 to 5, and the sum n₁+n₃ does not exceed 5, and
(uu) n₂ and n₄ are identical to or different from each other, are numbers from 0 to 4, and the sum n₂+n₄ does not exceed 4.

Examples of aurones are aureusidin, sulphuretin, hispidol as aglycon form and their glycosylated form as 6-glucoside-hispidol.

Preferably the monosaccharide may be substituted or unsubstituted glucose, rhamnose, galactose, arabinose, and xylose. Preferably the oligosaccharide may be the sugar moiety of the following flavonoids: tiliroside, orientin, schaftoside, saponarine, rutin, hesperidin, and diosmin or a polymer of one or more monosaccharide(s) previously described.
Flavanol (IX): 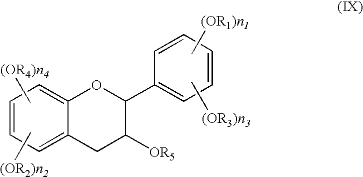
wherein:
(vv) the (OR₁), (OR₂), (OR₃) and (OR₄) groups are anywhere on the ring,
(ww) R₁ and R₂ are identical to or different from each other and represent a hydrogen atom, a saturated or unsaturated, linear or branched alkyl radical (C₁-C₆), a saturated or unsaturated, linear or branched acyl group with 1 to 6 carbon atom, a monosaccharide or an oligosaccharide,
(xx) R₃, R₄ and R₅ are identical to or different from each other and comprise a ω-substituted acyl group, or a monosaccharide or an oligosaccharide having at least one or more ω-substituted acyl groups, preferably from 1 to 6 acyl groups and more preferably from 1 to 3 acyl groups,
(yy) n₁ and n₃ are identical to or different from each other, are numbers from 0 to 5, and the sum n₁+n₃ does not exceed 5, and
(zz) n₂ and n₄ are identical to or different from each other, are numbers from 0 to 4, and the sum n₂+n₄ does not exceed 4.

Examples of flavanol (flavan-3-ols) are catechin, epicatechin, fisetinidol as aglycon form and their glycosylated form as catechin-7-O-xyloside, cyanidin-3-O-rutinoside, pelargonidin, and malvin.

Preferably the monosaccharide may be substituted or unsubstituted glucose, rhamnose, galactose, arabinose, and xylose. Preferably the oligosaccharide may be the sugar moiety of the following flavonoids: tiliroside, orientin, schaftoside, saponarine, rutin, hesperidin, and diosmin or a polymer of one or more monosaccharide(s) previously described.
Hydroxycoumarin (X): 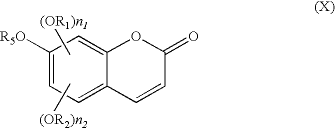
wherein:
(aaa) the (OR₁) and (OR₂) groups are anywhere on the ring,
(bbb) R₁ represents a hydrogen atom, a saturated or unsaturated, linear or branched alkyl radical (C₁-C₆), a saturated or unsaturated, linear or branched acyl group with 1 to 6 carbon atom, a monosaccharide or an oligosaccharide,
(ccc) R₂ and R₅ are identical to or different from each other and comprise a ω-substituted acyl group, or a monosaccharide or an oligosaccharide having at least one or more ω-substituted acyl groups, preferably from 1 to 6 acyl groups and more preferably from 1 to 3 acyl groups, and
(ddd) n₁ and n₂ are identical to or different from each other, are numbers from 0 to 3, and the sum n₁+n₂ does not exceed 3.

Examples of hydroxycoumarins are esculetin, umbelliferone, scopoletin, fraxetin as aglycon form and their glycosylated form as esculin, cichoriine, and fraxin.

Preferably the monosaccharide may be substituted or unsubstituted glucose, rhamnose, galactose, arabinose, and xylose. Preferably the oligosaccharide may be the sugar moiety of the following flavonoids: tiliroside, orientin, schaftoside, saponarine, rutin, hesperidin, and diosmin or a polymer of one or more monosaccharide(s) previously described.

Preparation of the Flavonoid Esters

The flavonoid esters according to the invention may be synthesized using known acylation processes from the state of the art. The acylation can be performed using an enzymatic process as described in the recently filed patent application no. EP 02292969.9 (Cognis France). The esters can also been obtained by chemical acylation methods. Chemical acylation agent may be chosen among acids of formula RCOOH, the halogen derivatives of these acids RCOHa1, anhydrides of formula RCOOCR or esters of formula RCOOR′ wherein R′ is a C1-C6 alkyl group, in anhydric appropriate solvent under inert atmosphere. Appropriate solvents may be chosen from the group consisting of toluene, pyridine, chloroform, tetrahydrofurane and acetone.

### EXAMPLES

### Example 1

### Synthesis of Ester of Rutin with Octadecandioic Acid

This reaction was carried out in a 250 ml batch reactor. Rutin (0.85 g, 1.4 mmol) and octadecandioic acid (0.97 g, 3.1 mmol) were dissolved in 250 ml tert-amyl alcohol. The medium was heated at 60° C. under vacuum (170 mbar). The formed vapor was condensed and recycled to the reactor throught a column filled with molecular sieves (50 g). This procedure allowed a low water level (<100 mM) in the reactor after 21 h. 2.5 g of the lipase of Candida antarctica (Novozym 435), a lipase immobilized on a macroporous acrylic resin with an activity of 7000 PLUg-1 (Propyl Laurate Synthesis), was then added.

After 70 h the enzyme was recovered by filtration. The medium was then concentrated by evaporation of solvent. To eliminate the residual substrates, two systems of extraction were used. A mixture of acetonitrile/heptane (3/5 v/v) is used to remove the palmitic acid, while the separation of rutin was carried out by an extraction with water/heptane (2/3 v/v).

The ¹H NMR of the ester obtained was:

¹H NMR: (400 MHz, DMSO d₆): 0.76 (d, 3H), 1.2 (m, 24H), 1.44 (m, 4H), 2.17 (m, 4H), 3.1-3.5 (broad, 8H), 3.7 (d, 1H), 4.45 (s, 1H), 4.65 (t, 1H), 5.44 (d, 1H), 6.19 (d, 1H), 6.36 (d, 1H), 6.83 (d, 1H), 7.5 (m, 2H) ppm.

### Example 2

### Synthesis of Ester of Rutin with Hexadecandioic Acid

The acylation of rutin (0.8 g, 1.3 mmol) with hexadecandioic acid (0.98 g, 3.4 mmol) was carried out as described in example 1.

After 63 hours reaction time the same procedure of purification by liquid-liquid extraction as described in example 1 allowed the recovery of rutin hexadecandioate.

The ¹H NMR of the ester obtained was:

¹H NMR: (400 MHz, DMSO d₆): δ 0.75 (d, 3H), 1.2 (m, 22H), 1.45 (m, 4H), 2.16 (m, 4H), 3.1-3.7 (broad, 11H), 4.45 (s, 1 H), 4.64 (t, 1 H), 5.43 (d, 1H), 6.18 (d, 1H), 6.36 (d, 1H), 6.84 (d, 1H), 7.50 (m, 2H), 12.6 (s, 1H, OH) ppm.

### Example 3

### Synthesis of Ester of Rutin with Azelaic Acid

The acylation of rutin (0.8 g, 1.3 mmol) with azelaic acid (0.58 g, 3.1 mmol) was carried out as described in example 1.

After 55 hours reaction time the enzyme was filtered. The medium was then concentrated by evaporation of solvent. The ester was recovered by two systems of extraction. A mixture of water/heptane (2/3 v/v) was used to removed azelaic acid, the recovery of the ester was carried out by extraction with ethyl acetate.

The ¹H NMR of the ester obtained was:

¹H NMR: (400 MHz, DMSO d₆): δ 0.75 (d, 3H), 1.24 (m, 12H), 1.48 (m, 8H), 2.20 (m, 8H), 3.15-3.50 (broad, 8H), 3.68 (d, 1H), 4.46 (s, 1H), 4.65 (t, 1H), 5.43 (d, 1H), 6.19 (d, 1H), 6.37 (d, 1H), 6.84 (d, 1H), 7.50 (m, 2H), 12.6 (s, 1H, C₅-OH) ppm

### Example 4

### Synthesis of Ester of Rutin with 11-mercaptoundecanoic Acid

The acylation of rutin (0.7 g, 1.2 mmol) with 11-mercaptoundecanoic acid (0.7 g, 3.1 mmol) was carried out as described in example 1.

After 64 hours of reaction time the enzyme was filtered. The solvent was then evaporated and the product was dissolved in methanol. The ester is recovered by two systems of extraction. A mixture of water/heptane (2/3 v/v) is used to remove acid, the recovery of the ester was carried out by extraction with dichloromethane.

The ¹H NMR of the ester obtained was:

¹H NMR: (400MHZ, DMSO d₆): δ 0.76 (d, 3H), 1.04 (d, 1H), 1.2 (m, 24H), 1.5 (m, 4H), 1.6 (m, 2H), 2.15 (m, 2H), 2.28 (m, 1H), 2.50 (m, 1 H), 2.68 (m, 2H), 3.1-3.9 (broad), 4.45 (s, 1H), 4.55 (m, 1H), 4.65 (t, 1H), 5.07 (d, 1H), 5.12 (d, 1H), 5.28 (d, 1H), 5.44 (d, 1H), 6.2 (s, 1H), 6.37 (s, 1H), 6.84 (d, 1H), 7.46 (m, 2H)

### Example 5

### Acylation of Naringin with Octadecandioic Acid

The acylation of naringin (0.59 g, 1 mmol) with octadecandioic acid (0.98 g, 3.1 mmol) was carried out as described in example 1.

After 50 h reaction time the same procedure of purification by extraction as described in example 1 allowed the recovery of the ester.

### Example 6

### Synthesis of Ester of Esculin with Octadecandioic Acid

The acylation of esculin (0.42 g, 1.2 mmol) with octadecandioic acid (0.97 g, 3.1 mmol) was carried out as described in example 1.

After 50 h reaction time the same procedure of purification by extraction as described in example 1 allowed the recovery of ester.

The structure was confirmed by ¹H NMR:

¹H NMR: (400 MHz, DMSO d₆): 1.2 (m, 24H), 1.5 (m, 4H), 2.2 (m, 4H) 3.15-3.55 (broad, 2H), 3.61 (t, 1H), 4.11 (dd, 1H), 4.34 (dd, 1H), 4.84 (d, 1H), 6.2 (d, 1H), 6.8 (s, 1H), 7.3 (s, 1H), 7.83 (d, 1H) ppm.

### Example 7

### Synthesis of Ester of Esculin with Thioctic Acid

The acylation of esculin (0.87 g, 2.5 mmol) with thioctic acid (1.23 g, 6 mmol) was carried out as described in example 1.

After 70 hours reaction time the enzyme was filtered. The medium was then concentrated by evaporation of solvent. The ester was recovered by two systems of extraction. A mixture of water/heptane/acetonitrile (2/3/0.4 v/v/v) was used to remove thioctic acid, the recovery of ester was carried out by extraction with dichloromethane.

The structure was confirmed by ¹H NMR.

¹H NMR: (400 MHz, DMSO d₆): 1.2-1.9 (broad, 8H), 2.1-2.4(broad, 4H), 3.2 (m, 2H), 3.5 (m, 1H), 3.7 (m, 1H), 4.12 (dd, 1H), 4.35 (d, 1H), 4.85 (d, 1H), 5.23 (d, 1H), 5.33 (d, 1H), 6.26(d, 1H), 6.84 (s, 1H), 7.33 (s, 1H), 7.86 (d, 1H) ppm.

### Example 8

### UVA Cytophotoprotection, Anti-oxidative Effect

The cytoprotection against UVA irradiation has been evaluated by a test on human fibroblasts because UVA radiation penetrates through the epidermis until the dermis where it induces oxidative stress, mainly by activation of photosensitising biological components, which catalyse the formation of ROS like anion superoxide, hydrogen peroxide and singlet oxygen, and lipoperoxydation of the cell membrane. These oxidative stress effects are evaluated in vitro due to measuring of the level of released MDA (malondialdehyde) and of intracellular GSH (reduced glutathion) (Morliere P., Moisan A., Santus R., Huppe G., Mazière J. C., Dubertret L.: UV-A induced lipid peroxydation in cultured human fibroblasts Biochim. Biophys. Acta (1991) 1084, 3:261-269).

The lipoperoxides formed after UVA irradiation undergo a decay into malondialdehyde which can form cross-links between many biological molecules like proteins with inhibition of enzymes and nucleic bases with risk of mutagenesis. Glutathione (GSH) is a peptide produced by the cells to protect them from oxidative stress or certain pollutants like mercury or lead. An increase in the GSH level enhances the activity of glutathion-S-transferase, a detoxification enzyme. GSH is evaluated according to the method of Hissin (Hissin P. J., Hilf R. A fluorometric method for determination of oxydised and reduced Glutathione in tissues. Analytical Biochemistry (1977) vol 74, pp 214-226).

Human fibroblasts were inoculated with growth medium (DMEM+FCS) and incubated 3 days at 37° C., with 5% CO₂. The growth medium was then exchanged with medium containing an ingredient to be tested and incubated 2 days at 37° C. with CO2=5%. After an exchange of medium with balanced salt solution, the cell culture was irradiated by UVA 20 J/cm². Cell proteins and GSH were measured, and MDA released in the supernatant was determined spectrophotometrically.

TABLE 1
Results in % against control (mean on 2-3 assays in triplicata):

| | | | | Cell |
|---|---|---|---|---|
| | Dose | MDA | Cell | GSH/protein |
| Product | % w/v | released | proteins | ratio |
| Control (not | — | 0 | 100 | 100 |
| irradiated) | | | | |
| Control/UVA | — | 100 | 107 | 78 |
| (20 J/cm2) | | | | |
| Rutin * | 0.003 | 79 | 126 | 72 |
| | 0.01 | 72 | 128 | 71 |
| Rutin | 0.001 | 46 | 139 | 73 |
| octadecandioate | 0.003 | 15 | 133 | 122 |
| according to | | | | |
| example 1 | | | | |
| Rutin | 0.003 | 46 | 129 | 101 |
| hexadecandioate | 0.01 | 21 | 178 | 75 |
| according to | | | | |
| example 2 | | | | |
| Dirutin | 0.001 | 39 | 138 | 98 |
| hexadecandioate | 0.003 | 9 | 149 | 154 |
| according to | | | | |
| example 10 | | | | |
| Mixture of Rutin | 0.001 | 48 | 142 | 92 |
| hexadecandioate | 0.003 | 25 | 143 | 153 |
| and Dirutin | | | | |
| hexadecandioate | | | | |
| according to | | | | |
| example 10 | | | | |
| Rutin azelaiate | 0.001 | 78 | 144 | 67 |
| according to | 0.003 | 64 | 165 | 64 |
| example 3 | | | | |
| Rutin 11- | 0.001 | 34 | 94 | 131 |
| mercaptoundecanoate | 0.003 | 0 | 89 | 283 |
| according to | | | | |
| example 4 | | | | |

* Rutin was purchased from Sigma.

The UVA irradiation has induced a release of MDA and a decrease of cell GSH. After incubation of the fibroblast with esters of rutin, a strong protection of cells against UVA-induced MDA released and GSH decrease was obtained, whereas rutin had very poorly protected the fibroblasts.

### Example 9

### UVB-cytophotoprotection and Anti-inflammatory Effect

The arachidonic cascade is an important mechanism of cutaneous inflammation. This cascade may be induced by several factors, particularly by UVB irradiation. UVB induces the inflammatory response by activation of phospholipase A2 (PLA2), which results in a release of arachidonic acid from cell membranes. Then other specific enzymes (so called cyclo-oxygenases) transform arachidonic acid in active components called prostaglandin (PG) which are secreted from the cells. The fixation of certain prostaglandins (PGE2) on specific skin receptors is followed by redness and swelling on human skin. On cultured human cells, these UVB effects on cell's membrane are associated with a release of a cytoplasmic enzyme into the supernatant medium: Lactate Dehydrogenase or LDH.

Human keratinocytes were inoculated with growth medium (DMEM+FCS) and incubated 3 days at 37° C. and 5% CO₂. The growth medium was then exchanged with balanced salt solution containing the ingredient to be tested, the cell culture was irradiated by UVB 50 mJ/cm² (DUKE GL40E lamp). After 1 day of incubation at 37° C. with 5% CO2, LDH and PGE2 released in the medium were determined, and cellular DNA was measured using a fluorescent probe to determine the cell viability.

TABLE 2
Results in % against control (mean on 2-3 assays in triplicata):

| | Dose | Keratinocytes | LDH | PGE2 |
|---|---|---|---|---|
| Product | % w/v | DNA | released | released |
| Control | — | 100 | 0 | 0 |
| (not irradiated) | | | | |
| Control/UVB | — | 23 | 100 | 100 |
| (50 mJ/cm2) | | | | |
| Rutin* | 0.03 | 69 | 17 | 0 |
| | 0.1 | 73 | 18 | 10 |
| Rutin | 0.001 | 23 | 73 | 28 |
| octadecandioate | 0.003 | 49 | 31 | 3 |
| according to | | | | |
| example 1 | | | | |
| Rutin | 0.003 | 24 | 53 | 2 |
| hexadecandioate | 0.01 | 39 | 19 | 0 |
| according to | | | | |
| example 2 | | | | |
| Dirutin | 0.001 | 38 | 44 | 3 |
| hexadecandioate | 0.003 | 35 | 33 | 1 |
| according to | | | | |
| example 10 | | | | |
| Mixture of Rutin | 0.0003 | 36 | 59 | 27 |
| hexadecandioate | 0.001 | 37 | 38 | 1 |
| and Dirutin | | | | |
| hexadecandioate | | | | |
| according to | | | | |
| example 10 | | | | |
| Rutin | 0.003 | 51 | 45 | 28 |
| azelaiate | 0.01 | 53 | 27 | 19 |
| according to | | | | |
| example 3 | | | | |
| Rutin 11- | 0.0001 | 44 | 75 | 26 |
| mercaptoundecanoate | 0.0003 | 41 | 92 | 12 |
| according to | | | | |
| example 4 | | | | |

*Rutin was purchased from Sigma.

The UVB irradiation has induced an inflammation with a release of PGE2 and with cell membrane injury as demonstrated by the release of LDH activity in the medium, and a decrease of keratinocytes cell number (decrease of around 77% of cell DNA). After incubation of the keratinocytes with rutin or the esters of rutin with ω-substituted fatty acid, and UVB irradiation, an increase of viable cells and a decrease of released LDH and PGE2 was obtained. But the esters of rutin are effective at doses 3-100 times lower than the active doses of rutin. These results demonstrate the anti-inflammatory efficacy of the tested products and their ability to protect cells from the damages induced by the UVB irradiation.

### Example 10

### Synthesis of Diester of Rutin with Hexadecandioic Acid:Rutin-C16 Diacid-rutin

This reaction was carried out in a 250 ml batch reactor. Rutin (10 g, 16.4 mmol) and hexadecandioic acid (4.2 g, 14.8 mmol) were dissolved in 250 ml tert-amyl alcohol. The medium was heated at 80° C. under vacuum (400 mbar). The formed vapor was condensed and recycled to the reactor through a column filled with molecular sieves (50 g) overnight. This procedure allowed a low water level (<100 mM) in the reactor. 7.5 g of the lipase of Candida antarctica (Novozym 435) was then added.

After 72 h the enzyme was recovered by filtration. The medium was then concentrated by evaporation of solvent. The medium is a mixture of rutin (10.4%), hexadecandioic acid (6.4%), rutin hexadecandioate (45.1%), dirutin hexadecandioate (38.1%). The purification by preparative HPLC allowed the separation of rutin hexadecandioate (rutin-O—(C═O)—(CH₂)₁₄—COOH) as characterised in example 2, of dirutin hexadecandioate (rutin-O—(C═O—(CH₂)₁₄—(C═O)—O-rutin), and of their mixture.

The ¹H NMR of the dirutin hexadecandioate obtained was:

¹H NMR: (400 MHz, DMSO d₆): δ 0.75 (d, 6H), 1.2 (m, 22H), 1.43(m, 4H), 2.13 (m, 4H), 3.1-3.7 (broad, 22H), 3.7 (d, 1H), 4.45 (s, 2H), 4.64 (t, 2H), 5.43 (s, 2H), 6.18 (s, 2H), 6.35 (s, 2H), 6.84 (d, 2H), 7.50 (m, 4H), 12.6 (s, 2H, OH) ppm.

### Example 11

### Solubility in Hydrophylic and Lipophilic Solvent

The solubility was determined by HPLC measurement after stirring during 1 hour at room temperature.

TABLE 3

| | Solubility | Solubility | |
|---|---|---|---|
| | in octyl- | in butylene | Solubility |
| Product | dodecanol | glycol | in water |
| Rutin* | 0.03 g/L | 22.6 g/L | 0.16 g/L |
| | 0.05 mM | 37.1 mM | 0.27 mM |
| Rutin hexadecandioate | 0.13 g/L | 39.4 g/L | 0.38 g/L |
| according to example 2 | 0.15 mM | 44.7 mM | 0.43 mM |
| Dirutin hexadecandioate | 0.03 g/L | >138 g/L | 0.58 g/L |
| according to example 10 | 0.02 mM | 94 mM | 0.39 mM |
| Rutin 11- | 0.15 g/L | 54.5 g/L | not |
| mercaptoundecanoate | 0.19 mM | 67.2 mM | determined |
| according to example 4 | | | |

The derivatives esters of the flavonoids have a higher solubility than the rutin in lipophilic and hydrophilic solvents as octyl-dodecanol, butylene glycol or water.

### Example 12

### Anti-free Radical Activity

Free radicals (FR) are reactive chemical species, characterised by non conjugated free electron. FR can appear from unsaturated lipids, certain amino-acids and above all from oxygen during spontaneous biological mechanism such as respiratory chain in mitochondria, or during natural biological process such as inflammation. Oxidative stress like UV or chemical pollutants induces also the rise of free radicals which provokes damages on all cellular and tissue constituents (lipids, proteins, sugars and nucleic bases) of living organisms. Indeed the FR toxicity is deeply enhanced by oxygen level and constitute a key process in ageing, in the appearance of serious diseases such as cancers, diabetes etc.

The anti-free radical (anti-FR) activity has been evaluated by biochemical tests to address the potential for scavenging superoxide anion (O2°). The O2° appears mainly from lipoxygenase activity, displayed by leukocytes along the leukotriens synthesis from arachidonic acid released during inflammatory process (Bouclier M & Hensby C N. Prostaglandines et leucotrienes en physiologie cutanee. Bulletin d'Esthétique Dermatologique et de Cosmétologie, (1986) pp 17-22).

Lipoxygenase was incubated with a specific substrate (unsaturated fatty acid) and the flavonoid esters. Then the rate of released superoxide anions was determined using Luminol luminescent probe to calculate the IC₅₀ (mean of 2 assays).

| Product | IC₅₀ (w/v). |
|---|---|
| Rutin octadecandioate according to example 1 | 0.0034 |
| Rutin hexadecandioate according to example 2 | 0.0036 |
| Dirutin hexadecandioate according to example 10 | 0.0028 |
| Rutin azelaiate according to example 3 | 0.0025 |

What is claimed is:

1-14. (canceled)

15. A flavonoid ester with a ω-substituted C6 to C22 fatty acid, wherein the ω-substituted C6 to C22 fatty acid is a saturated or unsaturated, linear or branched aliphatic C6 to C22—carboxylic acid having one or more polar groups.

16. The flavonoid ester according to claim 15, wherein the flavonoid is an aglycone or the glycosylated form of a polyphenol selected from the group consisting of a flavone, a flavonol, a flavanone, a flavanol, a flavanolol, an isoflavone, an anthocyanin, a proanthocyanidin, a chalcone, an aurone and a hydroxycoumarin.

17. The flavonoid ester according to claim 15, wherein the polar group is on the terminal carbon atom of the C6 to C22—carboxylic acid.

18. The flavonoid ester according to claim 15, wherein the polar group of the ω-substituted C6 to C22 fatty acid is a derivative of a carboxylic acid selected from the group consisting of a carboxylic acid (COOH); an amide (CONR′₂ or CONR′₃⁺S⁻) wherein R′ is a hydrogen atom, a saturated or unsaturated, linear or branched alkyl C1-C6 radical, or an aryl, aralkyl or aralkylene radical and S⁻ is a counter ion; a COHa1 wherein Ha1 is a halogen atom; and a COSH.

19. The flavonoid ester according to claim 15, wherein the ω-substituted C6 to C22 fatty acid is dicarboxylic.

20. The flavonoid ester according to claim 19, wherein the ω-substituted C6 to C22 fatty acid is selected from the group consisting of octanedioic acid, azelaic acid, decandioic acid, dodecandioic acid, hexadecandioic acid and octadecandioic acid.

21. The flavonoid ester according to claim 15, wherein the ω-substituted C6 to C22 fatty acid is a dicarboxylic acid linked to a flavonoid by an ester bond on one of its carboxylic groups (HOOC—X—C(═O)—O-flavonoid), wherein X is a saturated or unsaturated, linear or branched alkyl radical (C₄-C₂₀).

22. The flavonoid ester according to claim 15, wherein the ω-substituted C6 to C22 fatty acid is 11-mercaptoundecanoic acid or thioctic acid.

23. The flavonoid ester according to claim 15, wherein the polar group of the ω-substituted C6 to C22 fatty acid is a thiol or an alkylthioalkyl group.

24. The flavonoid ester according to claim 15, wherein the ω-substituted C6 to C22 fatty acid has two adjacent polar groups selected from the group consisting of diol, dithiol, 1,2-dithiane, 1,3-dithiane and epoxide.

25. A nutritional or cosmetic or pharmaceutical composition containing a flavonoid ester according to claim 15.

26. A nutritional or cosmetic or pharmaceutical composition comprising liposomes or microcapsules containing a flavonoid ester according to claim 15.

27. A nutritional or cosmetic or pharmaceutical composition according to claim 25, containing 0.0001 to 10 wt % of a flavonoid ester.

28. The flavonoid ester according to claim 15 incorporated into a cosmetic preparation as an agent to protect skin and scalp against damage caused by UV radiation, mitochondrial or nuclear DNA damage caused by UV radiation, and aging, or as an anti-inflammatory and/or soothing and relieving agent.

29. The flavonoid ester according to claim 15 incorporated into a preparation for stimulating the metabolism and the immune defense of human skin, including defense against oxidative or environmental stress or pollutants, for a dermatological anti-inflammatory care preparation, or for a draining, veinotonic or slimming preparation.

30. The flavonoid ester according to claim 28, wherein the ester is used in the preparation in quantities of 0.0001 to 10 wt % based on the final composition.

31. The flavonoid ester according to claim 28, wherein the ester is present in the preparation in the form of liposomes or microcapsules.

32. The flavonoid ester according to claim 29, wherein the ester is used in the preparation in quantifies of 0.0001 to 10 wt % based on the final composition.

33. The flavonoid ester according to claim 29, wherein the ester is present in the preparation in the form of liposomes or microcapsules.
